(19) United States
(12) Patent Application Publication
(10) Pub. No.: US 2002/0168784 A1
(43) Pub. Date: Nov. 14, 2002

(54) Agglutination assays

(76) Inventors: Erling Sundrehagen, Oslo (NO); Dag Bremnes, Oslo (NO); Geir O. Gogstad, Oslo (NO)

Correspondence Address: SCHWEGMAN, LUNDBERG, WOESSNER & KLUTH, P.A., P.O. BOX 2938, MINNEAPOLIS, MN, 55402, US

(21) Appl. No.: 09/768,040

(22) Filed: Jan. 23, 2001

Related U.S. Application Data
(63) Continuation of application No. PCT/GB99/02398, filed on Jul. 23, 1999.

(30) Foreign Application Priority Data
Jul. 23, 1998 (GB) 9816088.0

(51) Int. Cl. G01N 33/536; G06F 19/00; G01N 33/48; G01N 33/50; G06K 9/00

(52) U.S. Cl. 436/536; 702/19; 702/21; 382/128

(57) ABSTRACT

A diagnostic system comprises a desk-top, flat-bed optical scanner (101) which scans a substrate, such as a microtitre plate (107) containing a mixture (105) of a sample and an agglutination reagent which react to generate an assay result of an agglutination assay. The scanner (101) generates a digitized image of the assay result. A personal computer (103) coupled to the scanner (101) is arranged to perform an analysis of the digital image to provide a quantified result for the degree of agglutination of the assay result.

The invention relates to apparatus and a method for analysing agglutination assays and in particular provides a diagnostic system usable in a laboratory or, especially, at the point-of-care, e.g. in a physician's office.

Many diagnostic assays are available nowadays to physicians, and an increasing number do not require him to send the patient's sample (e.g. blood, urine, saliva, stool) to a diagnostic laboratory for analysis. Such in-office assays enable a result to be obtained rapidly and entered on to the patient's computer record by the physician or his assistant.

One particularly useful form of assay is an agglutination assay in which a sample is mixed with one or more agglutination reagents. Bonding sites on the agglutination reagent(s) bond to corresponding sites on components of the sample, if present, and this bonding results in agglutinates, which are visible clusters of bonded reagent and sample component. Thus, a desired reagent may be mixed with a sample and the presence of agglutinates in the mixture indicates the presence of the corresponding component in the sample.

Traditionally, agglutination assays have been carried out qualitatively, with a judgment being made by the laboratory technician as to a positive or negative result. However, we have realised that a quantitative result can be obtained from an agglutination assay by analysis of the assay result to give a quantified result for the degree of agglutination, rather than a simple positive or negative result.

Furthermore, we have now found that a quantified result can be obtained in a simple and straightforward fashion by the use of an imaging device (e.g a desk-top, flat-bed optical computer scanner) capable of generating a digitised record of the image, i.e. the assay result, produced by an agglutination assay and of software capable of performing analysis of the digital image by manipulation (analysis) of the digitised record.

Thus viewed from one aspect, the invention provides apparatus for the analysis of an agglutination assay comprising:

an imaging device arranged to generate a digital image of an assay result comprising a mixture of a sample and at least one agglutination reagent; and

data processing means arranged to process said digital image to generate a quantitative result representative of the degree of agglutination of the sample and reagent.

According to the invention therefore a quantified result for the agglutination assay may be achieved simply and easily, and reflects the degree of agglutination rather than a simple yes/no result. Furthermore, the quantified result can easily be transferred to other data processing systems, for example to a patient data file for the patient providing the sample.

Viewed from a further aspect, the invention provides a method for the analysis of an agglutination assay comprising the steps of:

generating a digital image of an assay result comprising a mixture of a sample and at least one agglutination reagent; and

processing said digital image to generate a quantitative result representative of the degree of agglutination of the sample and reagent.

Preferably, the imaging device is a desk top, flat bed computer scanner, as this provides a low-cost imaging device which is readily available. More preferably, the data processing means comprises a personal computer, as this is again low-cost and readily available.

The digital image may be a monochrome image. This would provide acceptable results for example in the case of agglutination assays involving white or light agglutinates imaged against a black or dark background. Preferably, the digital image is a digital colour image. In this way, greater flexibility is provided in distinguishing the agglutinates from the background. Furthermore, agglutinates of two or more different colours formed by two or more different agglutination reagents reacting with the same sample in the same assay result may be identified so that two tests may be carried out simultaneously.

More generally therefore and, viewed from a yet further aspect, the invention provides a method for performing an agglutination assay comprising the steps of:

providing a sample;

providing at least two agglutination reagents, each having different optical properties;

mixing the sample and the reagents to form an assay result;

generating a digital image of the assay result; and

processing said digital image by reference to the optical properties of each reagent to generate a quantitative result representative of the degree of agglutination of the sample and each reagent.

The optical properties may be any suitable property, for example fluorescence, colour, degree of light scattering, shape, size or texture of the resultant agglutinates etc. Preferably, the optical properties are the colours of the reagents (or the resultant agglutinates).

The assay result will generally be formed in or on a substrate. A suitable substrate is for example a glass or plastics plate, such as a microscope slide or a microtitre plate, or similar substrate. Preferably, means are provided on the substrate to enclose the assay result within a defined area for ease of identification of the assay result in the digital image and to maintain a consistent depth of the assay result for a predetermined volume of sample and reagent(s).

Preferably, digital image data corresponding to the assay result within the digital image is located automatically, for example by a suitable arrangement of the data processing means.

Generation of the quantitative result may involve determining at least one statistical characteristic of the distribution of pixels within the digital image. Suitable characteristics are mean pixel level, standard deviation, higher order statistical moments, auto-correlation, fourier spectrum, fractal signature, local information transform, grey level differencing etc.

In one arrangement, generation of the quantitative result may involve determining the proportion of an area, preferably only the area of the assay result, of the digital image representative of agglutination products. Thus, for example, the background colour may be identified and the foreground colour (corresponding to the agglutinates) may also be identified and the proportion of the area of the image, or that region of the image corresponding to the assay result, being of the foreground colour may be calculated.

Generation of the quantitative result may involve locating within the digital image clusters of contiguous pixels which are representative of agglutination products. Such clusters may be identified as groups of pixels having all their neighbouring pixels of the same, foreground, colour. The quantitative result may be generated by reference to the area, for example total area, of the clusters, the distribution of the clusters in the digital image or the number of the clusters in the digital image.

The apparatus (system) of the invention may and preferably will be arranged to analyse assay results from a plurality (i.e. two or more) of different assays.

The data processing means may be a personal computer. For example, a desk-top or lap-top (or palm top etc.) or other relatively inexpensive machine, e.g. of the type produced by Apple, Dell, Compaq, Olivetti, IBM and many others. Alternatively however a more powerful or extensive computer system may be used, especially where the system is located within a hospital or commercial organization (in which case the imaging device may be linked directly or indirectly, e.g. telephonically, to a component of a computer network). Indeed even with “personal” computers the connection to the imaging device may be indirect, e.g. telephonic. The results generated by the system and method of the invention are preferably entered directly into the relevant patient's computer file, for example on the PC, or on a central computer to which the PC is linked by a network, or on a remote computer via a permanent or impermanent linkage (e.g. via the internet, etc.). In general, the system and method of the invention are intended primarily for use in the clinician's office/laboratory or in a hospital diagnostics laboratory and so direct entry into the patient's file on the PC itself or on a network-linked computer is of particular interest.

The desk top scanner and/or the PC used in this system may be standard products available on the personal computer and computer accessories market. The scanner may operate in reflectance or transmission mode and in the latter instance may be a transparency (i.e. slide or dia) scanner or a transparency scanner add-on to a larger bed scanner. One example of a scanner that may be used is the Relisys Infinity or the Hewlett Packard ScanJet 6100C. This can be used to assign pixels to a grey scale or alternatively to assign a colour value (e.g. green, blue and red combinations) to each pixel.

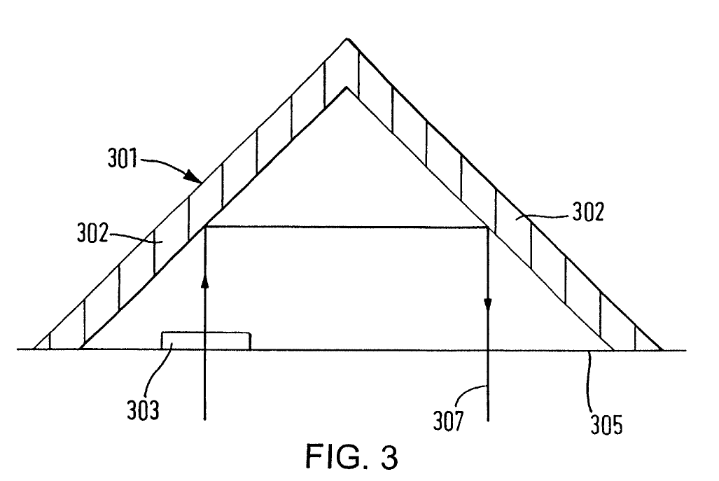

In order to use a transparent assay result with a standard, flat-bed scanner, an adapter may be used, for example, as shown in FIG. 3. A suitable adapter 301 comprises two perpendicular, flat mirrored surfaces 302 which are placed over the assay result 303 on the scanner glass 305 such that they each make an angle of 45° with the scanner glass. Light 307 from the scanner passes vertically out through the glass (and thus through the assay result) and is reflected into a horizontal path by one mirror. The horizontal light is then reflected back towards the scanner glass by the second mirror. Thus, the scanner can detect an image of the light transmitted by the assay result in a position adjacent the assay result.

The invention is not, however, limited to an arrangement comprising a flat-bed scanner and a personal computer. For example, a digital camera may be used to generate the required digital image data. Furthermore, a video camera arranged to generate digital image data, for example by means of a frame grabber, may be used. Each of these devices is readily available to the medical practitioner.

In general, the imaging device will be arranged to scan the assay result under the illumination of daylight or a white light source. For example, in the case of a flat-bed scanner, white light is generated by the scanner itself. However, in the case of a digital camera or video camera, the white light source may be external to the imaging device and may be simply the ambient lighting in the medical practitioner's office. In such cases, where the light source is not controlled by the imaging device, it is advantageous for calibration to take place. Thus, the digital image data may comprise data corresponding to the colour composition of a calibration object of a predetermined colour or colour(s). The calibration object may be presented to the imaging device together with the assay result or may be presented to the imaging device in a calibration step. In either case, it is possible for the data processing means to compare the digital image data relating to the calibration object with stored data relating to the predetermined colour(s) of the calibration object and thereby determine a relationship between the colours and the digital image data. This relationship, which may be in the form of a look-up table or an algorithm, may then be used to translate the digital image data relating to the assay result into normalised digital image data that is independent of the characteristics of the light source and the imaging device.

The calibration object, or an additional calibration object, may also be used to calibrate the magnification of the imaging device. For example, the calibration object may be provided with a region of predetermined spatial dimensions from which the data processing means may calculate a relationship between the dimensions represented-by the digital image data and the actual dimensions of the objects represented thereby. Alternatively, the imaging device may be maintained in a fixed spatial relationship with the plane in which the image result is or will be located. This is generally the case with a flat-bed scanner, but a suitable jig or the like may be provided for a digital camera or video camera.

The system of the invention may be used in combination with appropriate photodetectors and/or illumination to quantify the properties of analytes exhibiting fluorescence and/or phosphorescence. Analysis could also be carried out beyond the visible spectrum, for example in the infra-red or ultra-violet regions.

Information found in grey-scale or colour images can be collected and stored to file in digitised form using flatbed scanners, digital cameras or video cameras. The bit-depth of the stored digitised file (standard bit-values: 1, 4, 8, 15, 32) will determine the amount of information that can be retrieved. The number of shades of grey or colour stored in these files are found as exponentials of 2, i.e. bit-depth 1(2¹), 2(2²), 3(2³), 15(2⁵ of each of red, green and blue colour), 24(2⁸ of each of red, green and blue colour). This means that 1, 4 and 8 bit files contains 2, 16 and 256 shades of grey respectively. Similarly a 15 bit file contains information about 32768 different colours (2⁵=32 different shades of each of the red, green and blue colour), and 24 bit files information about 16777216 different colours (2⁸=256 different shades of each of the red, green and blue colour). A bit depth of 32, possible to obtain even with simple flatbed scanners, makes it possible to store additional information of the colour intensities of each of the collected colours found in a 24 bit file. In more detail, this means that the last 8 bits are utilised for recording intensity, resulting in 256 different intensities (2⁸=256) for each of the 16777216 different colours stored.

As a consequence of the information stored in the digitised files, quantitative measurement of colour is possible. Using a 10 bit file, 1024 different shades of grey are available. A digitised image of a single spot of ink on white paper is measured as a high intensity black centre with edges along the rim composed of low intensity shades of light grey/white. This information can be presented and visualised as a three-dimensional bell shaped surface with the third dimension expressed as intensity of black. Integrating across the surface gives the volume of the body covered by this surface. This volume can then be used as a direct quantitative measure when comparing different spots with different intensities. Similarly, using 15, 24 or 32 bit files it is also possible to derive quantitative information regarding the colour composition of the original image. Colour measurements and quantification measuring spots of either pure red, green or blue colour is easy and equivalent to the measurement performed using grey scale data. One way of doing this is to transfer the recorded colour data by matrix calculations to hue-saturation values (HS-values). However, quantification of mixtures of colours are more complicated.

The optical part of flatbed scanners contains three different detectors each with spectral sensitivity to the three primary colours of light, i.e. red, green and blue, respectively. x(λ) has a high sensitivity in the red wavelength area, y(λ) in the green wavelength area and z(λ) in the blue wavelength area. The colours that we perceive and which are recorded are all the result of different x(λ), y(λ) and z(λ) proportions (stimuli) in the light received from an object. The resulting three values X, Y and Z being recorded are called tristimulus values. In this system every perceived and recorded colour can be expressed with its unique co-ordinate (X,Y,Z) in a co-ordinate system where the axes are formed by the three basic colours red, green and blue. Different numerical expressions have been developed to express colour numerically. In a photometer/refectometer used in analytical chemistry to record colours and intensity, monochromators or multiple sensors are used to measure the spectral reflectance of the object at each wavelength or in each narrow wavelength range. Simpler instruments, like flat bed scanners, as previously described measure colour by reflectance measurements only at the wavelengths corresponding to the three primary colours of light (red, green and blue). The three different reflectance values recorded (tristimulus values) can then be used to convert the data to colour spaces like the “Yxy” , “L*a*b” or the “L*c*h” systems. Digital cameras and video cameras are also capable of producing a digital output for each pixel in a digital colour image composed of the X, Y and Z values (RGB values) for that pixel. Thus, the output from such cameras may be used interchangeably with the output of a flat-bed scanner for the purposes of the invention.

Measurements of mixtures of different colours using flat bed scanners or similar imaging devices result in multivariate systems in terms of quantification of each of the colours in the mixture. Colours will be recorded as blends of each of the basic colours red, green and blue. A mixture of two different colours, e.g. red and blue, may be recorded as a new colour with its own intensity. In digitised form this colour will be determined by the relative amount of each of the two chromophores used and characterised by its tristimulus values (X,Y,Z), the basis for all quantitative information stored. To quantify the relative relationship between red and blue in a spot composed of two colours, information regarding the specific colour recorded for the mixture is sufficient. Using flatbed scanners in colour mode and a sufficient bit depth of the digitised data, quantitative information can be achieved. However, to be able to perform the quantification of each of the colours in the mixture, standard solutions with known concentration must be used. Standards of two different colours and their mixtures can be spotted on a white surface, measured and used to establish standard curves for the determination of the composition of an unknown colour spot composed of the same two colours.

The complexity of the quantification process measuring colours will vary depending upon the spectral characteristics of the chromophores used. This is because only three different wavelength areas are used in the recording process using flat bed scanners. The possibility of separating different chromophores then depends upon the spectral separation of the different chromophores involved and their absorption maxima relative to the sensitivity of the x(λ), y(λ) and z(λ) detectors of the scanner. The basis for separating different chromophores is that the reflectance from each of the chromophores used (e.g. two or three) is different for at least one of these three wavelength areas. For optimal chromophore systems, i.e. where the spectroscopic overlap at x(λ), y(λ) and z(λ) can be neglected, the corresponding X, Y or Z co-ordinate value can be used for their quantification. If chromophores with spectral overlap are used, all three values must be used as part of a multicomponent treatment of the recordings related to concentration. As an example, a blue and red chromophoric system with optimal spectral properties, the relative amount of red and blue chromophore can be calculated by measuring the average X/Z-ratio for every pixel in the recorded spot. By this way every mixture of these two chromophores can be recorded and estimated using a flat bed scanner or similar image acquisition device.

The relationship between the assay result and the colour image data may be stored in the form of a look-up table or an algorithm. In general, this relationship will be specific to a particular assay type and/or substrate.

Thus, for maximum flexibility, the data processing system will have access to a plurality of relationships corresponding to the plurality of substrates that may require analysis. These relationships may be stored locally to the data processing system or may be stored remotely, in which case the data processing system may access the relationships by means of a network or other communication channel. In the case of remote storage of the relationships, a database of relationships may be maintained and updated centrally, for example by the manufacturer of the assay substrates. In this way, the latest analysis relationship will always be available to the medical practitioner.

Advantageously, the data processing means of the invention is arranged to automatically identify the assay result within the digital image data and thereby locate the areas of interest in the image data.

Thus, the assay result may be located in the digital image data according to the following method of analysing a digital image of a scene comprising at least one object, the object comprising at least one field, corresponding to the assay result. The method comprises:

identifying the location of said object in said image;

classifying said object;

identifying digital data corresponding to said field by reference to stored data relating to said classified object and the location of said object;

converting said digital data to a corresponding quantitative result.

The object, which may correspond to the substrate on or in which the assay result is contained, may be classified by geometric parameters, such as length, width, radius etc., by comparing identified parameters with corresponding geometric parameters for known objects.

The substrate may be associated with a machine-readable identifier, for example a bar code, or similar machine-readable coding, the identifier including information relating to the assay type and preferably also the associated patient. Preferably, the identifier will be optically readable by the imaging device. However, it would also be possible for the identifier to be readable by a separate data acquisition device, for example a bar code scanner, magnetic strip reader, smart card reader or any device capable of converting data stored on the identifier to digital data which can be passed to the data processing system. In a simple form, the identifier may include a single number which corresponds to a record of a type of assay or a particular patient in a database accessible to the data processing system. However, the identifier may contain more information, which may or may not be associated with additional information available to the data processing system.

Agglutination reactions are valuable analytical tools which can be applied to many reaction systems in which multivalent binding between reactants is possible. Typical examples are immunoassays which may be generally:

mixing polyclonal antibodies with a sample containing an antigen corresponding to the antibodies, and observing the formation of immunoagglutinates

mixing a monoclonal antibody with a sample containing an antigen carrying at least two antigenic functions (bivalent or multivalent antigen) and observing the formation of immunoagglutinates

mixing at least two different monoclonal antibodies with a sample containing a monovalent antigen and observing immunoagglutination

any of the reactions mentioned above, but applying the antibodies coupled to particles, such as latex particles, colloids, etc.

any of the reactions mentioned above, but applied to antigens present on cell surfaces in which case the number of antigens per physical unit is normally a hundred or more, and in which case such cells may be agglutinated by monoclonal antibodies even if each antigen molecule is monovalent.

The reactions are typically observed on the surface of a solid substrate such as a glass or plastic plate, or in a solution in a microtitre plate. The solid surface is preferably coloured to contrast with the colour of the agglutinate.

The formation of agglutinates is dependent on the concentration of antigen in the sample. Thus, the more antigen present in the sample, the more frequent and larger the agglutinates. However, at a certain concentration level the antibodies will saturate the antigenic binding sites. When the number of antigen binding sites exceeds the number of antibody binding sites, the increase in agglutination will be correspondingly less pronounced and completely disappear at very high antigen levels. Thus, the level of reactants should be adjusted to take this aspect into consideration.

Agglutination reactions may also be performed with any sets of molecules binding to each other, provided that each of the reactants has at least two binding sites each, or is coupled to a particle or otherwise linked together so that two or more binding sites per physical unit is created. Examples of other systems than antibodies/antigens that may form agglutinates are (poly)carbohydrates/lectins, biotin or biotinylated compounds/avidin or streptavidin, corresponding sequences of nucleic acids, any protein receptor and its corresponding ligand etc.

Although the agglutination reactions are, in fact, quantitative in nature, such that the level of agglutination corresponds to the presence of an analyte in a sample, the interpretation of the result is traditionally merely qualitative. Since many of the analytes which may be the subject of such agglutination reactions are desired to be measured quantitatively, other and more complicated methods like ELISA, RIA, immunofiltration or immunochromatography methods have been used.

Agglutination-based products for detection and quantitation of analytes have been produced for a wide range of analytes. Very early on, the field was developed using products for the detection of human chorionic gonadotropic hormone (HCG) in urine, for the diagnosis of pregnancy. Two different principles were used:

1. products were made with antibodies on a particle surface, which gave agglutination in the presence of the analyte; and

2. products were made with antigen on the surface of the particles, and reagent containing antibodies was added together with the test sample.

In this second variant, agglutination took place in the absence or at low concentration of the analyte. However, a higher concentration of the analyte occupied the antibodies and hindered the agglutination.

Agglutination tests for slides and visual inspections were made, and some companies, including Technicon, made autoanalyzers based upon instrumental measurements of particle inspection by measurement of particle number and particle size. Furthermore, a long list of reagents for measurement of analytes by means of the measurement of alteration in turbidimetry as a function of the agglutination have been made. Automated spectrophotometers with a capacity for many hundred of tests per hour, e.g. Hitachi Instruments from Boehringer Mannheim in Germany and Cobas instruments from Roche in Switzerland, uses such reagents. These instruments, however, are very large and less convenient for patient-proximate testing and smaller laboratories and offices.

Typical protein analytes for agglutination technology are C-reactive protein (CRP), transferrin, albumin, pre-albumin, haptoglobin, immunoglobulin G, immunoglobulin M, immunoglobulin A, immunoglobulin E, apolipoproteins, lipoproteins, ferritin, thyroid stimulation hormone (TSH) and other proteinaceous hormones, coagulation factors, plasminogen, plasmin, fibrinogen, fibrin split products, tissue plasminogen activator (TPA), beta-microgobulins, prostate-specific antigen (PSA), collagen, cancer markers (e.g. CEA and alpha-foetoprotein), several enzymes and markers for cell damage (e.g. myoglobin and troponin I and T).

Furthermore, agglutination reagents for testing for drugs, including prescription drugs and most illegal drugs, and many non-proteinaceous hormones, such as testosterone, progesterone, oestriol, have been made.

Moreover, many agglutination test kits for infectious diseases have been made, including mononucleosis, streptococcus infection, staphylococcus infection, toxoplasma infection, trichomonas infection, syphilis. Such reagents and reagent sets are either based upon detection of the infectious agent itself, or detection of antibodies produced by the body as a reaction to the infectious disease.

It should be noted, however, that the examples given above are not considered to be a complete listing of the applications of agglutination assays and many other applications are possible.

Applying an imaging device, such as a flat bed scanner, to the reading of agglutination reactions will introduce a quantitative aspect to such reactions.

The imaging device, e.g. flat bed scanner, may be applied to the measurement of simple contrast since agglutinates normally occur as white spots formed out of a transparent solution. Such spots may be easily visualised or measured against a dark background. However, such direct agglutination is less frequently used since the reactions are not as easily controlled as when the antibodies are coupled to particles. In most cases, white latex particles are used, and the occurrence of white aggregates against a background of fully dispersed white latex may be less easy to visualise or read. Thus, colours are preferably applied to the particles. Colours are preferably chosen to facilitate the distinction between background and agglutinates.

Another possible aspect of this is to apply particles that change colour compared to the background when agglutinated. An example of such reactions is the agglutination of metal colloids. Most such colloids change colour upon agglutination, for example, colloidal gold is reddish in its original form, turning to blue when the agglutinates exceed a certain size, and further to black when the agglutinates become even larger.

Another possibility is to mix particles of two different colours, for example blue and yellow particles, of which only one type, say the yellow particles, contain the antibodies. Thus, the unreacted solution will appear green while the introduction of an antigen will lead to the formation of yellow agglutinates towards a background changing from green to blue.

A further possibility is that of reading two or more reactions simultaneously. In the above example, if the blue and yellow particles are coupled to two different antibodies, respectively, each antibody being directed towards different antigens, the original green solution will form a mixture of yellow and blue aggregates if contacted with a solution containing both antigens. A flat bed scanner may easily measure the occurrence of each type of aggregate, independently of each other, and thus provide a quantitative result for two simultaneous reaction in one single reaction. Furthermore, such reactions may of course be conducted with a plurality of differently coloured particles, each containing antibodies directed towards different antigens.

The agglutination reactions should be performed either by mixing the sample and reagent(s) on a flat surface and measuring the agglutination, or the reaction may be conducted in a test tube or a reaction chamber followed by pouring the reaction mixture to a surface after a certain time. The surface is preferably transparent in order to allow light from the flat bed scanner to interact with the reaction mixture. However, the surface may also be coloured in a way that an optical filter is created in order to facilitate reading of certain wavelength intervals of light.

The surface may be shaped so that the reaction mixture is enclosed within a distinct region in order to improve reproducibility in quantitative readings. This may be achieved by a circular elevation in a plastic surface which can be made according to standard production methods, or by the use of a microtitre plate.

Furthermore, a device in which an agglutination reaction to be read by a flat bed scanner is performed, may conveniently also contain a cover which may be tilted over the reaction zone before reading. This will protect the flat bed scanner from being contaminated by the reaction mixture. Furthermore, such a cover may be coloured in order to form a proper background for optimal reading of the agglutination assay.

Some embodiments of the invention and some examples will now be described by way of example only and with reference to the accompanying drawings, in which:

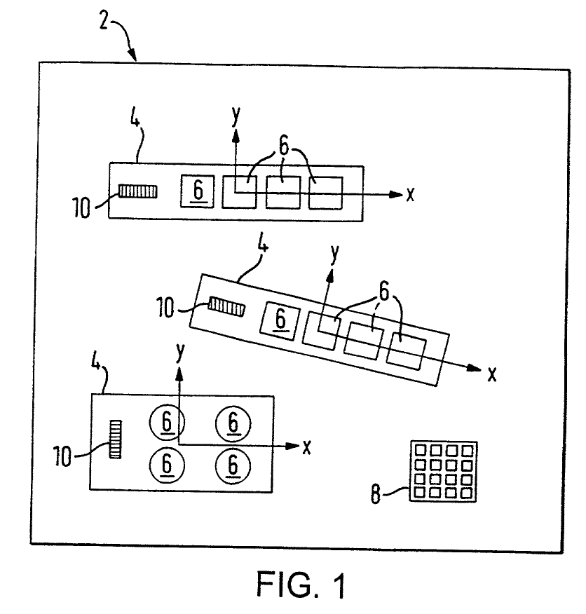

FIG. 1 is a schematic digital image produced according to the invention;

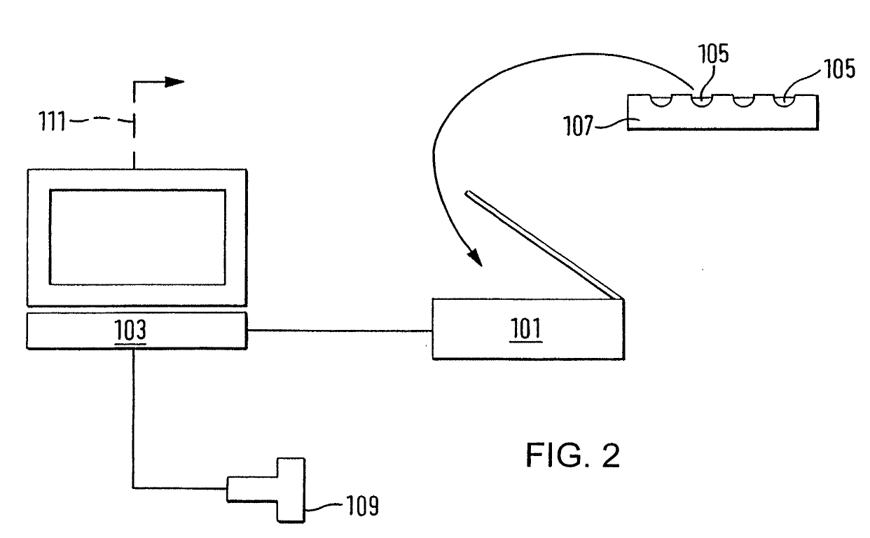

FIG. 2 is a schematic diagram of a PC and scanner arranged according to the invention;

FIG. 3 is a schematic view of an adapter used to enable a scanner to operate in a transmission mode;

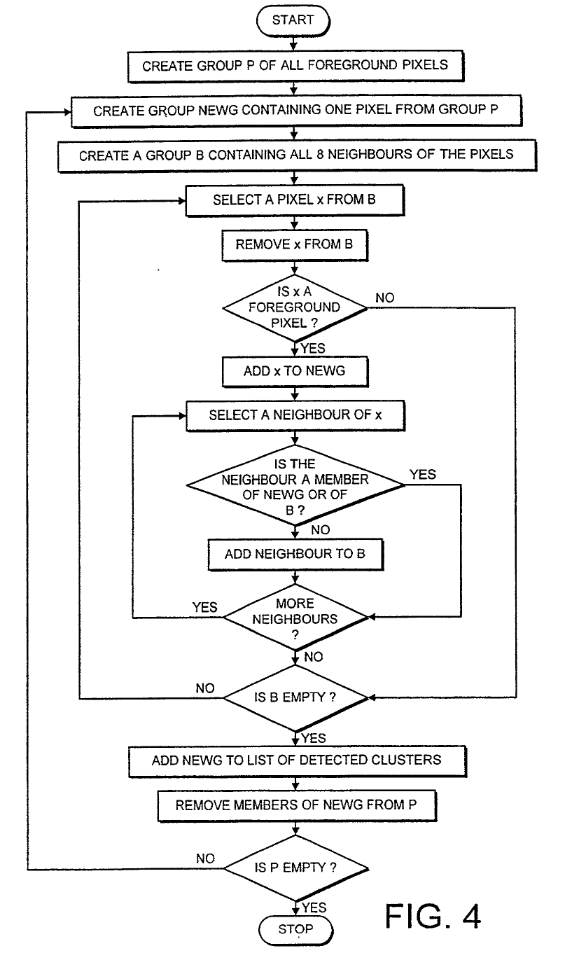

FIG. 4 is a flow chart showing a cluster identification algorithm;

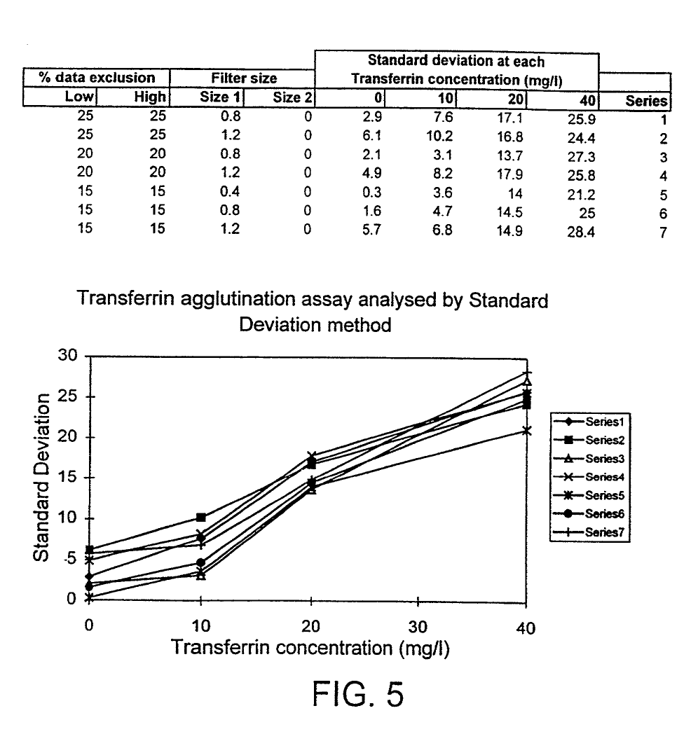

FIG. 5 shows the results of a transferrin agglutination assay analysed by a standard deviation method;

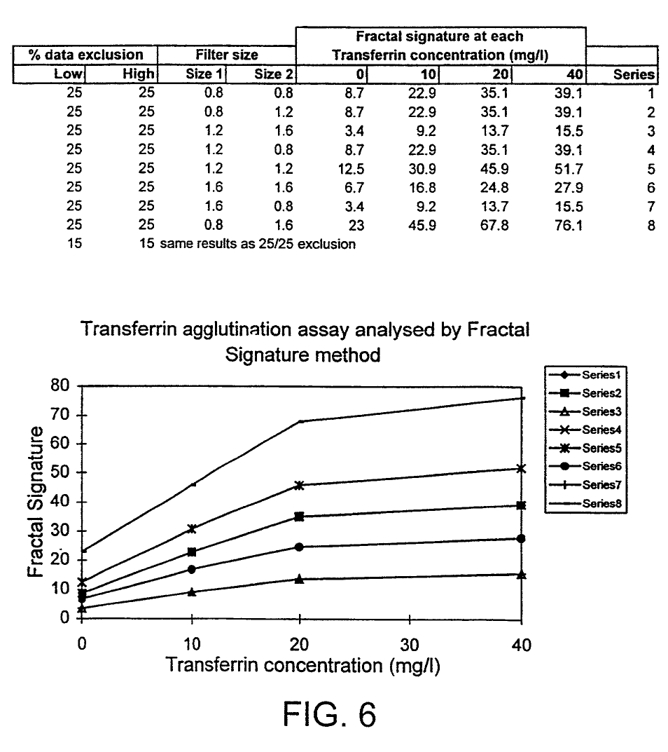

FIG. 6 shows the results of a transferrin agglutination assay analysed by a fractal signature method;

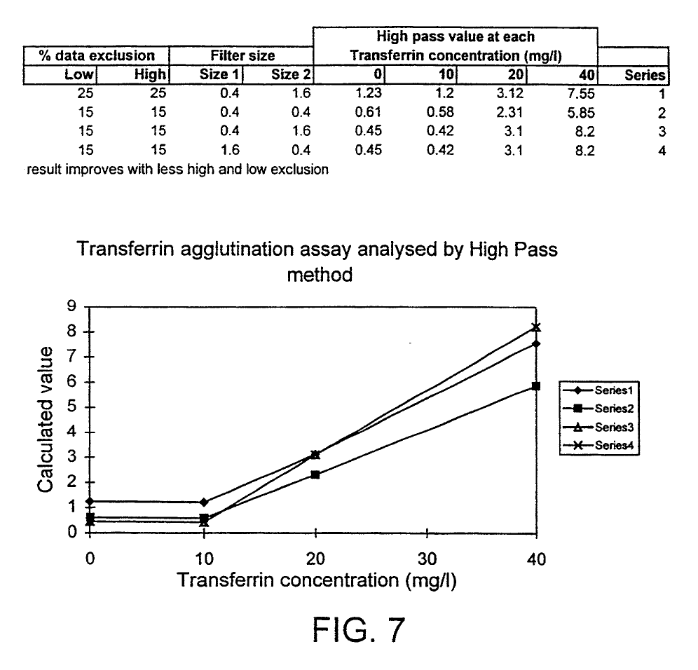

FIG. 7 shows the results of a transferrin agglutination assay analysed by a high pass method;

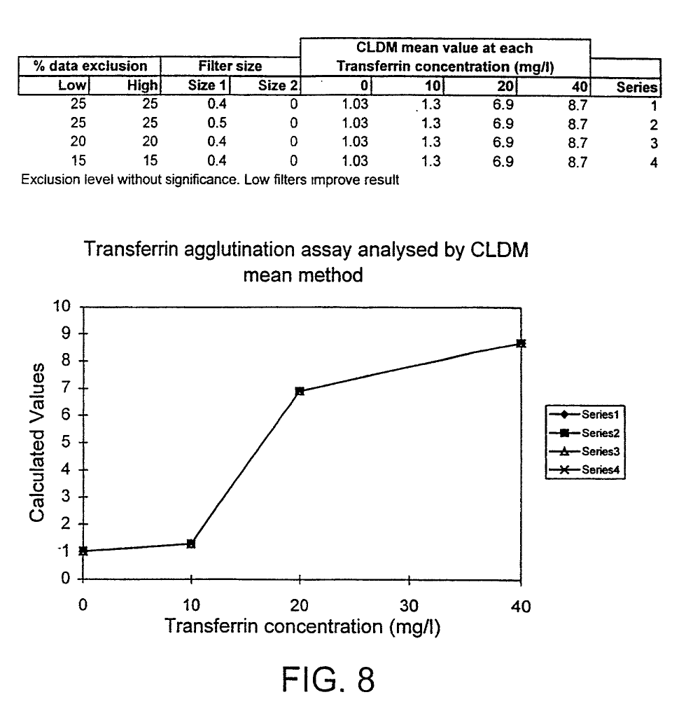

FIG. 8 shows the results of a transferrin agglutination assay analysed by a CLDM mean method;

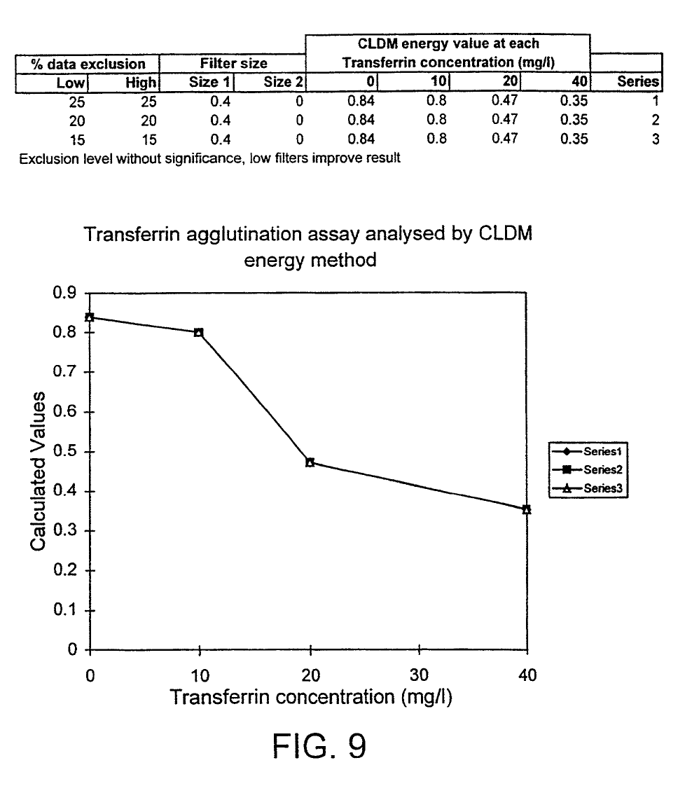

FIG. 9 shows the results of a transferrin agglutination assay analysed by a CLDM energy method;

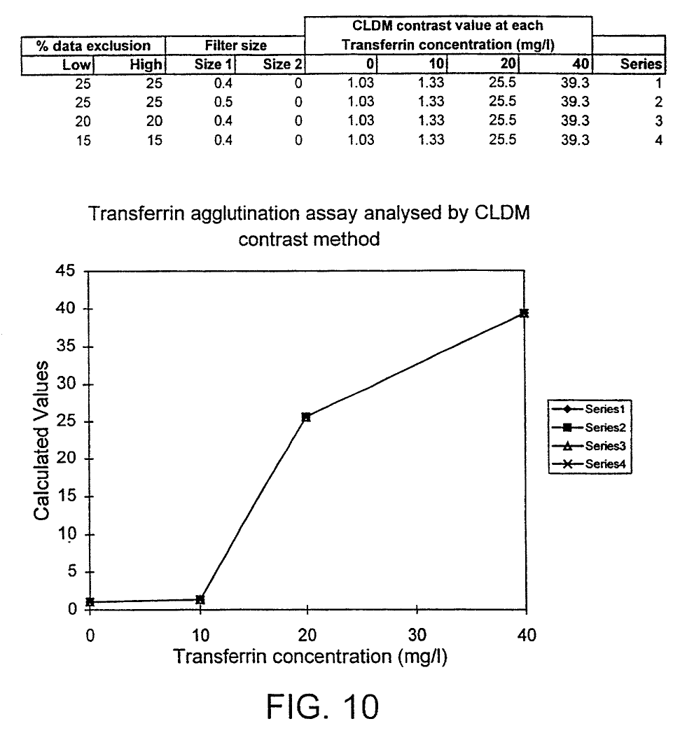

FIG. 10 shows the results of a transferrin agglutination assay analysed by a CLDM contrast method;

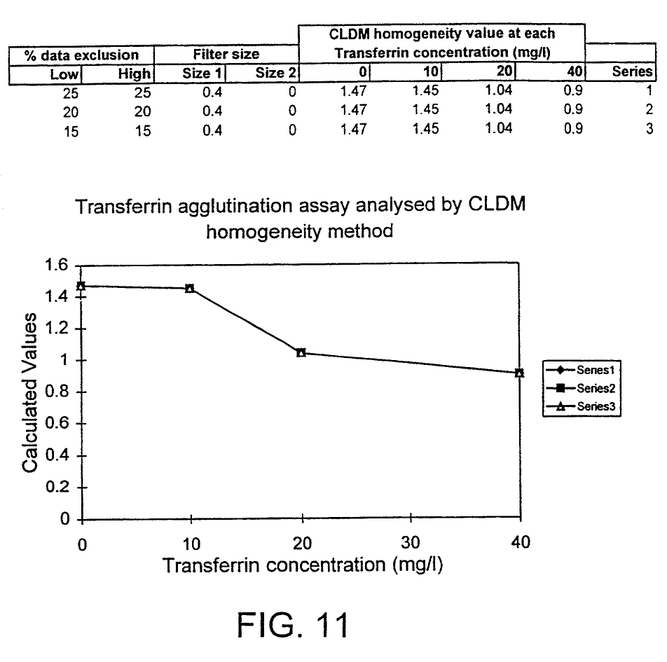

FIG. 11 shows the results of a transferrin agglutination assay analysed by a CLDM homogeneity method;

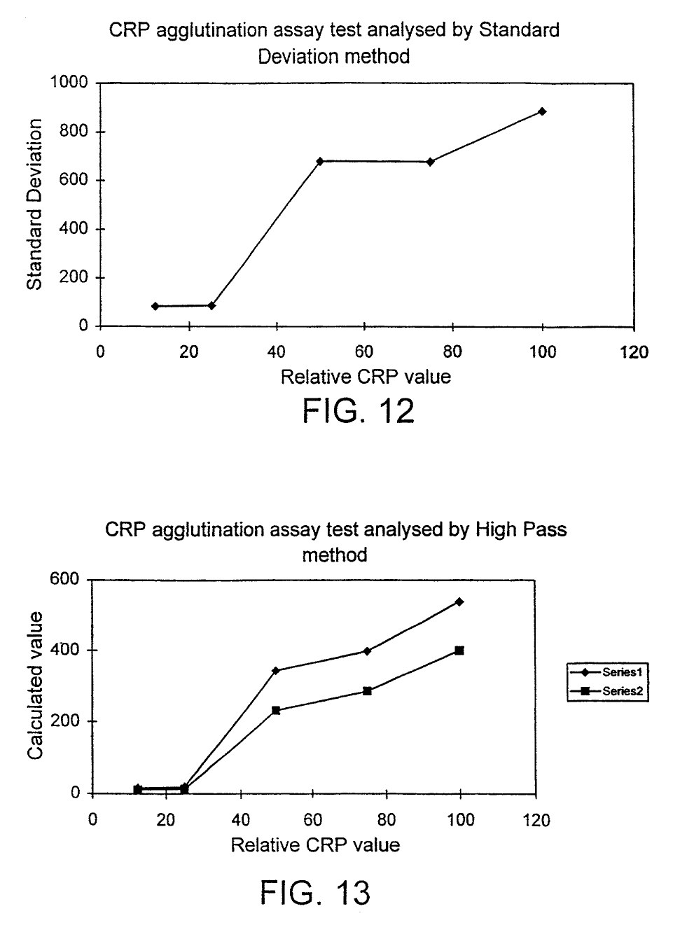

FIG. 12 shows the results of a transferrin agglutination assay analysed by a standard deviation method;

FIG. 13 shows the results of a CRP agglutination assay analysed by a high pass method;

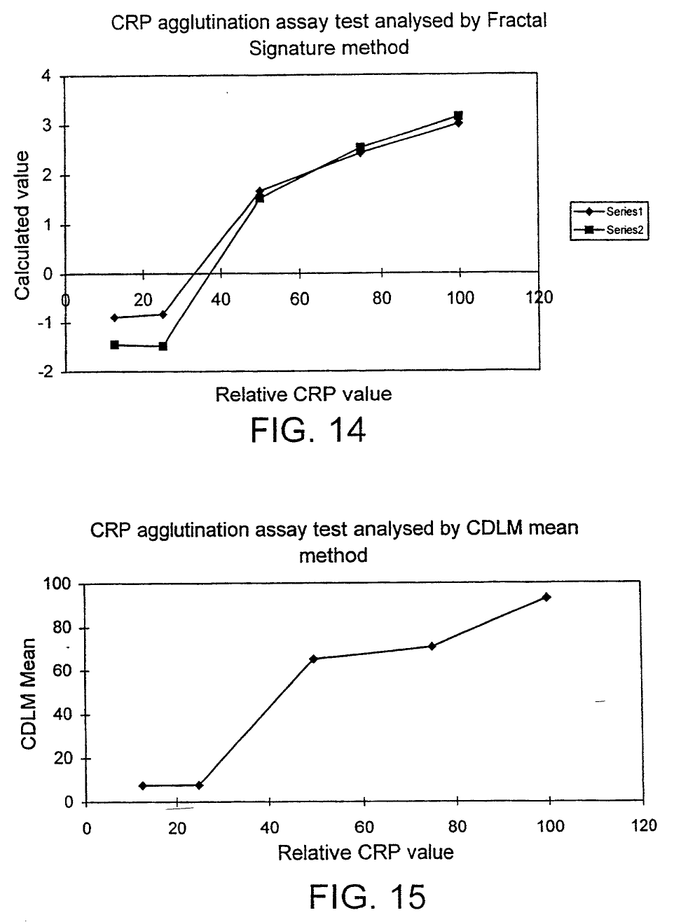

FIG. 14 shows the results of a CRP agglutination as say analysed by a fractal signature method; and

FIG. 15 shows the results of a CRP agglutination assay analysed by a CLDM mean method.

FIG. 1 shows schematically an exemplary digital image 2 produced by a scanner in accordance with the invention. The image 2 corresponds to an arrangement of objects 4 each of which contains one or more fields 6. In the following, such an arrangement of objects 4 will be referred to as a “scene”, the image 2 corresponding to the scene. Each of the objects may be, for example, a microscope slide or a microtitre plate or a similar flat substrate. The fields 6 within each object 4 are defined regions, where an assay result is expected to be located, for example the wells of a microtitre plate.

The scene also comprises a calibration object 8. The calibration object 8 is of a predetermined colour or colours, which colour or colours are known to the data processing system for analysing the image 2. Thus, variations in the ambient lighting conditions or in t he sensitivity of the photodetectors of the scanner between the production of subsequent images 2 can be compensated with reference to the calibration object 8. Suitable predetermined colours for the calibration object 8 are a grey scale (all greys from 0% to 100%) each shade of which will contain equal proportions of red, green and blue. The calibration object may be divided into identifiable fields each of a different grey shade or other predetermined colour. In an alternative arrangement, the calibration object may be replaced or supplemented by one or more calibration fields on each object 4.

Each object may also comprise an identification field 10, such as a bar code or other suitable machine-readable coding. The identification field 10 may contain information identifying the type of assay results in the fields, the sensitivity of the fields or other information relating to the object 4. The identification field 10 is generally provided at a predetermined location on the object 4 such that it can be easily located in subsequent analysis of the image 2 or used to define the accurate positions of the fields 6. The identification field 10 may be applied to the object 6 as part of the manufacturing process or may be applied once the assay has been carried out. In the former case, the identification field 10 may simply contain a serial number or a code (e.g. a bar code) by which the particular object may be identified during subsequent use. Thus, the data processing system used to analyse the image 2, may contain information associated with this serial number, and thus with the particular object 4. For example, the information may relate to the assay type, date and time of the assay etc. In the case of medical assays, the information may include data identifying the patient, such as name, are, sex, symptoms etc. If the identifying field 10 is applied to the object 4 after manufacture, the field itself may be used to store the information described above, thereby obviating the need for additional dedicated data storage. When the scene contains a plurality of objects 4 the identification field 10 may be used to differentiate between the objects and ensure that the correct results are associated with the correct object. In this way, the quantified assay result may be passed automatically to the correct patient file in a patient database.

As has previously been described, the data processing system for analysing the image 2 may be a personal computer. An example of a suitable arrangement of a personal computer and scanner is shown in FIG. 2. Scanner 101 is connected to PC 103. In order to produce an image for analysis, a predetermined volume of analyte and agglutination reagent is mixed in a well of a-microtitre plate 105 to form an assay result 107. The microtitre plate 105 is then placed on the scanner glass. The PC 103 is also connected to a bar code reader 109 for reading bar codes from patient records, substrates and analyte containers etc. The PC 103 has an optional data connection 111 to a remote computer for exporting quantified assay data.

Referring back to FIG. 1, the personal computer is provided with object data relating to the various types of objects 4 that it is required to analyse, including the calibration object 8. The object data will, in general, be supplied by the manufacturer of the objects 4 and will include, for each object: the geometrical dimensions of the object (e.g. width and height or for circular or elliptical objects radius or radii) together with the tolerances for those dimensions; the number, location on the object (with tolerances) and identification of the fields 6 provided on the object 4; and the location of the identification field 10. For each type of field 6, some of which may be provided on a number of objects 4, field data will also be provided including: an identification of the property that is indicated by the field 6; and a description of the relationship between the degree of agglutination in the field 6 and the property indicated by the field. The relationship between the degree of agglutination of the field 6 and the property indicated by that field may be stored in the form of an algorithm, for example dependent on the mean and standard deviation of the distribution of agglutination products with the indicated property. Alternatively, the relationship may be stored as a look-up table which maps the degree of agglutination of the field 6 on to the value of the property indicated by that field. The values stored in the look-up table may be determined empirically prior to the distribution of the objects for general use.

The image will generally be stored in 24 bit colour, i.e. 8 bits for each component colour, for example red, green and blue. Before analysis of assay results can be undertaken, the scanner should be calibrated. Such a calibration may be undertaken before every analysis or may be undertaken on installation of the scanner. The first step in the calibration is the production of an image corresponding to an empty scene, i.e. the scanner background which is preferably black. However, the background will not be perfectly black and dust or dirt deposits may result in blemishes on the background. The 24-bit empty image of an empty scene is converted to an 8-bit grey scale image by adding together the 8-bit red, green and blue values for each pixel and dividing the sum by three. The mean grey scale value is calculated for all pixels in the empty image. A grey threshold value is determined which is equal to the calculated mean grey scale value for the empty image plus a small offset, which may be, for example, a multiple or fraction of the standard deviation of the grey scale pixel distribution in the empty image. Thus, the grey threshold is deemed to be the value below which pixels may be considered to correspond to the scanner background.

The positions of pixels with high grey values in the empty image are stored, these pixels being deemed to be due to dirt on the scanner background, and are deleted from all subsequent images, so that the image is not distorted by these “dirty pixels”.

The second stage of the calibration is the calibration of colour reproduction of the imaging system and the data processing system using the calibration object 8. The calibration object 8 is identified as an object in the same way as objects to be analysed (as is described hereinafter), but is classified as the calibration object 8. The colours of the fields of the calibration object 8 determined by the data processing system are compared to the predetermined values for these colours, which are stored in the data processing system. On the basis of the differences in the determined colours and the expected colours, a calibration look-up table is calculated which maps the detected value of each colour component to its actual value. In the case of a flat-bed scanner, initially an image 2 may be processed which contains only the calibration object 8, so that the calibration look-up table can be constructed. As the variations in ambient light level will be insignificant for a flat bed scanner, there will be no need for re-calibration between subsequent images. However, the calibration object 8 can be included in every scene if variations in the light source or the sensitivity of the photodetectors are expected. In this case the calibration object 8 will be identified initially by the data processing system and the calibration look-up table will be constructed before the other objects 4 in the scene are processed.

In the first stage of processing an 8-bit grey image is created from the 24-bit colour image by summing the three 8-bit colour component (RGB) values for each pixel and dividing by three. Of course, the grey image may be created in any suitable manner, for example as a weighted average of the RGB values, rather than a simple average. This grey image is used in the identification of objects 4 and is not used in the analysis of the fields 6, where the 24 bit colour image is used. The dirty pixels identified in the calibration stage are removed from the image 2 by replacing their grey value with the mean value of their neighbouring pixels. The RGB values of the dirty pixels in the colour image are also respectively replaced by the mean RGB values of their pixels neighbouring the dirty pixel. This may be done before the grey image is created. The background in the grey image is removed by setting to zero the value of each pixel which has a detected grey value below the threshold calculated during the calibration stage.

Subsequently, unwanted gaps in the image are removed by operating on the grey image with a maximum operator and then with a minimum operator. A maximum operator is a matrix of n by n pixels, the function of which is to replace the central pixel of the matrix with the highest pixel value occurring within the n by n matrix. Similarly, a minimum operator replaces the central pixel of the matrix with the lowest value found therein. Each pixel of the grey image is operated on as the central pixel of the maximum/minimum operator. The size n of the operators is determined by the objects that are to be analysed. Objects that contain very dark regions (gaps) extending from one boundary to the other, or at least very close to the boundaries, will be considered as two objects by the data processing system as the gap will be indistinguishable from the background. Thus, by removing such gaps from the grey image it will be ensured that the objects are correctly identified by the data processing system. The gaps are not removed from the colour image, however. Thus the maximum gap size g to be removed from a particular image is the largest gap appearing in any of the objects in the image. The operator size n is equal to the maximum gap size g (in meters) multiplied by the resolution of the image (in pixels per meter). The maximum gap size g for each object is part of the object data stored in the data processing system for each object 4. The maximum gap size for a particular image 2 is the maximum gap size g for all objects which can appear in the scene. Thus, this may be the maximum gap size for the entire list of objects 4 stored in the data processing system or for a selected list of objects that has been defined by the operator as expected to be detected in the scene.

Once the dirty pixels, background and gaps have been removed in the pre-processing stage, the contours of each object 4 in the grey image are traced. Any objects having a boundary less than a predetermined threshold are deleted as being of no interest. This threshold may be determined with reference to the list of all objects stored in the data processing systems or a user-defined list of all objects that are expected to appear in the scene; When the boundary of each object has been determined, the centre of the object is calculated and the principal axes (x, y shown in FIG. 1) of the object 4 are determined. If, from the boundary, it is determined that the object is circular, any two perpendicular axes coincident at the centre of the object are chosen. If it is determined that the object is square or rectangular, axes x, y are chosen perpendicular to the sides of the object 4. In this way, a coordinate system is established for each object of interest with the origin of the coordinate system at the centre of the object. The length and width (or radius) of the object have also been determined from the boundary, so that the object can be classified by comparison of these parameters with the stored object data. If the object meets the criteria of more than one set of stored data, further features, such as field positions, of the object are identified and compared to stored data. The object is classified as the stored object type which it most closely matches, within an acceptable error range. If the object does not match the parameters for any of the object data, it is classified as an unknown object. The location of the fields within the classified object are known from the data stored in the data processing system in terms of the local coordinate system that has been determined. A complete set of data has now been created from the 8 bit grey image, which data identifies each object in the grey image (and thus in the colour image) and the exact location of each field (including the identification field 10) in that object. Thus, from the 24-bit colour image the RGB values for each field 6 of each object 4 can be retrieved. These RGB values can be converted to device-independent colour values using the calibration look-up table. In addition, the information from the identifying field 10 of each object can be read and associated with the assay values which will be calculated for that object. All identifying and assay data is in electronic form and therefore can be passed easily to a, for example patient, database or similar internal or external data system for association with other data relating to the assay, such as demographic or treatment data.

As will be seen from the above, a flat bed scanner can be used simply to obtain accurate assay information from an assay object. The image may be stored in a device-independent format so that it may be processed at a remote location or archived for future reference. For cleanliness and ease of handling, the objects may be placed on or in a window, holder or adapter, which may advantageously locate the object on the scanner.

However, the above processing methodology allows for the use of other data acquisition means, as there is no requirement for the accurate positioning or lighting of the objects. Hitherto, complex devices such as spectrophotometers have been used to ensure the accurate location of assay fields and the accurate reproduction of the colour of such fields. However, in accordance with the invention, accessible and relatively inexpensive digitisation equipment can be used to obtain the initial image data, which is then processed by the data processing system to obtain the assay results. Thus, as an alternative to a flat-bed scanner, a digital camera may be used to obtain the image data. In this case, the objects to be analysed are placed on a surface above which the camera is positioned. The scene is photographed by the digital camera to produce the digital image. The image may then be processed in the same way as for the image obtained by the scanner. However, in order to obtain accurate identification of the size of each object, data relating to the height of the camera above the surface and the camera angle may need to be made available to the data processing system. In addition, a calibration object may be required in each scene as the resultant image may be affected by ambient lighting conditions. The calibration object may also contain spatial calibration information such as one or more regions of predetermined dimensions. Similarly, as an alternative to the scanner or digital camera, a video camera and a frame grabber may be used to produce the digital image data.

An advantage of a digital camera or video camera over a flat-bed scanner is that the substrate may be located in the view of the camera without physical contact therebetween. In the case of a flat-bed scanner, the assay substrate is placed on the scanner glass and thus deposits, such as urine, faeces or blood, from the substrate may be transferred to the glass. However, a camera may be positioned at a distance from the substrate, for example above the substrate, and may accurately generate digital colour image data of the substrate without contacting the substrate.

Using, for example, a Cinet, 32 MB RAM, 166 MHz Pentium processor PC coupled to a Hewlett Packard ScanJet 5p colour scanner, the process of the invention may be performed using the following steps:

(A) The “scene” is configured

(B) The scan of the scene is performed

(C) The scene is segmented into “regions”

(D) The regions are identified

(E) The “quality” of the regions is checked

(F) Data values determined are associated with patient identifier information

(G) The data is exported to a central computer and into the appropriate patient file.

In step (A), if appropriate, the operator will set a scan delay (e.g. 60 or 120 seconds) and select whether the substrate is to be scanned once or more than once, e.g. twice or more.

The scan delay will generally cause appropriate prompt signals, e.g. audible beeps, to occur at pre-set delay times before the scan is performed. This allows the operator to effect the assay by mixing the sample and the agglutination reagent(s) and place the substrate on the scanner bed so that the scanning takes place at the desired time after the assay commences. This is important as many assay results must be read at a particular time after assay commencement. Where multiple substrates are to be read by the scanner, these will preferably be spaced apart on the scanner bed such that they are read by the scanner at the same time delay after the sample and reagent have been mixed. To assist in this, a mask may be placed on the scanner bed showing the operator where to place the substrate or substrates.

Multiple scans will be selected where it is desirable to follow the progress with time of the assay result, e.g. to report the peak value or to report the change in value over a specific time period. Multiple scans will also be selected where the substrate is arranged for a multiple assay, i.e. to provide values for more than one parameter characteristic. For example by having different agglutination reagents in different wells of a microtitre plate, where the individual assays involved require different development times.

Because the assays may require specific development times, it is preferred in the methods of the invention to use reading devices (e.g. scanners) which have uniform start-up times, i.e. which will read the substrate with the same time delay after instruction each time. For this reason, the HP ScanJet 5p has been found to be a preferred flat-bed scanner.

In step (A), the operator will generally also select the area to be scanned and select whether bar codes (or other machine readable codes) are allowed and optionally he will also select which such codes are allowed.

Moreover the operator may select whether or not a prompt signal is required and the timing and type of such a signal (e.g. audible or visible).

If bar codes are allowed, the data handling operation will involve identification of the bar code or codes associated with the substrate or substrates. This may for example serve to identify the patient and/or the nature of the substrate and hence the assay or assays involved. A patient bar-code may conveniently be provided on a tear-off portion of the label for the sample-container for the test substance. Such a tear-off portion can be attached to the substrate before scanning or placed adjacent to the substrate on the scanner bed. The substrate itself will preferably carry a code identifying the nature of the assay.

The PC will conveniently be set up to offer the operator a list of assays which it can analyse and from which to select the assays the operator is using. For the operator's convenience, where multiple substrates are being scanned, the operator will conveniently be able to specify whether all substrates derive from the same patient, whether all substrates are the same (i.e. perform the same assays), or whether a mixture of substrates is being scanned. Either before or after scanning, the operator will conveniently be prompted to identify the patient, e.g. by providing a code permitting the results to be exported to the patient's data file.

With this input from the operator the scanning may proceed.

If a prompt signal has been selected, the operator will wait for the prompt, mix the first sample(s) and reagent(s) in the first substrate on receiving the prompt and then place the substrate on the scanner bed in the assigned position after the required contact time, mix the second sample(s) and reagent(s) on receiving the next prompt, etc. until the scanner bed is fully loaded. After the predetermined period(s) from the first prompt the scanner will perform the first and any subsequent scans and export the image data to the PC.

The subsequent image data handling by the PC can be effected in many ways and that described hereafter is simply a preferred scheme.

(1) Find gap size

(2) Make a binary or gray image

(3) Find the “active” image

(4) Remove noise

(5) Run maximum operator in a first (x) direction

(6) Run maximum operator in a second orthogonal (y) direction

(7) Run minimum operator in x-direction

(8) Run minimum operator in y-direction

(It is possible to configure the scene to require the maximum and minimum operators to be run in one direction only. This saves time but restricts the location of objects.)

Gap size for the substrates is specified by the operator's identification of the nature of the substrate in step (A).

The PC takes the image data and segments the scene into regions. For each pixel of the colour image, the colour black is assigned if the mean value of the R, G and B values ((R+G+B)/3) is below a first threshold and the difference between the highest and lowest R, G or B values is not greater than a second threshold value. This produces a treated colour image and from this a grey scale image is created using the mean R, G, B values now assigned to the individual pixels. For example this may be achieved by scanning an empty image, i.e. a clean and empty scanner bed, and setting the first threshold as the mean (R+G+B)/3 value for this empty image plus a pre-set value. The second threshold may be set as the product of a pre-set coefficient and the average value of the difference for the R, G and B values from the R, G and B values for the empty image. In other words, a pixel is not discarded if its average (R+G+B)/3 value is below the first threshold but one or two of its R, G and B values are individually noticeably higher than the respective “background” R, G or B value.

From this grey image, the active area, the area containing the substrates and/or bar codes, is selected by moving inwards from the image edges until the number of non-black pixels exceeds a preset limit. The noise may be removed by setting a noise size as half the gap size and removing all structures smaller than the noise size, i.e. setting to black all pixels in such structures. This reduces the possibility of a noise pixel being included in an object boundary. Gaps are then removed by operating on the image with a maximum operator followed by a minimum operator. The maximum operator is as wide as the largest gap size for the objects (substrates) allowed in the scene. Of course, if the largest gap size is zero this operation is not required.

The objects in the image are then located by finding a non-black pixel with an adjacent black pixel (i.e. a border pixel) and following the path of adjacent such non-black pixels until the original is returned to.

From the resultant list of border pixels, for each region the centre is calculated and the geometry is determined, e.g. as a rectangle or circle. Travelling from the centre of each region to its borders along its principal axes, the length and width of the region is calculated.

Each such region found by this segmentation step is then classified as an object or an unknown. The border data for the unknowns are combined to create regions which are classifiable as objects. For each object the length and width are compared with the length and width data of allowed objects (from the database stored by the PC which contains the characteristic data for the substrates it is set up to read). A quality factor is then determined for the orientation of each object and the orientation is selected as being that with the lowest (i.e. best) quality factor. For each object, the quality factors for all objects it is allowed to be is determined and the object is identified as being that with the lowest quality factor.

For each field in the object (located using the data for the allowable objects in the PC's object database mentioned above), the field centre is located. The position of the field is then fine-tuned by calculating for each R, G and B image the standard deviation for its fit to the allowable object when moved small distances Δx and Δy and selecting the position at which the standard deviation is minimised.

For pixel calibration, one may use a standard colour card to construct a table for RGB values. Using the same colour-card the same table should be constructed for the particular scanner being used, the colour space should be divided (e.g. mapped onto a 16×16×16 cube space), and each calculated or calibration point may be assigned into one such division (cube). For more precision, corrected positions of such points within each division may be interpolated from the values of the division corners (i.e. the corners of one of the 16³ cubes making up the colour space).

Once the fields have been located in the digital image, the pixels of each field are analysed to obtain a quantified result for that field.

For fields representative of an assay result in which agglutinates of one colour appear as foreground against a background colour of the agglutination mixture, each pixel is assigned to either the group of foreground pixels or background pixels. This is done by calculating the distance Db, Df of the RGB colour vector x of each pixel in RGB colour space from a predetermined mean background vector μb or mean foreground vector μf. The distances are calculated using the following formulae:

Db=(trans(x−μb))*(Inv(Σb))*(x−μb)

Df=(trans(x−μf))*(Inv(Σf))*(x−μf)

where Σ represents the covariance matrix, defined as:

Σb=E{(x−μb)*(trans(x−μb))}

and E is the expectation operator, trans is the transpose operator and Inv is the invert operator.

Thus, if for a particular pixel Df<Db the pixel is classified as a foreground pixel, i.e. the pixel represents an agglutinate, and if Df>Db the pixel is classified as a background pixel.

Next, the pixels are classified into sub-groups of each of the foreground and background groups, where each subgroup represents a cluster of connected pixels. A cluster is defined as a group of pixels, where it is possible to move from one pixel in the group to any other without moving outside the group. The clusters are located from the group of foreground (or background) pixels using the algorithm shown in FIG. 4. According to this algorithm, pixels are selected sequentially from the group P of all foreground pixels. One pixel is selected from P and made the initial member of a new group newG. A group B of all 8 pixels which neighbour the selected pixel is created. Thus, if the selected pixel is (i,j) in Cartesian spatial co-ordinates, the neighbouring pixels are (i−1,j−1), (i,j−1), (i+1,j−1), (i−1,j), (i+1,j), (i−1,j+1), (i,j+1) and (i+1,j+1). A first pixel x is selected from group B and then removed from that group. If x is a foreground pixel it is added is to group newG. The 8 pixels neighbouring pixel x are then examined sequentially and any that are not already members of group B or group newG are added to group B. Thus, group B represents the group of pixels bordering the pixels of group newG and group newG is expanded by adding pixels from B if these pixels are foreground pixels. Eventually, group B will be empty because on the previous examination, the only additional neighbouring pixels were background pixels. At this point, it is known that group newG is surrounded by background pixels. Thus, group newG is added to the list of clusters and the pixels contained in group newG are removed from group P as it is now known that these pixels are members of cluster newG. The algorithm stops when group P is empty, i.e. all pixels have been classified into clusters.

Properties of the digital image and thus of the assay result can be calculated from the characteristics of the clusters. Suitable characteristics are:

total area, i.e. number of pixels, of foreground or background;

total area of foreground or background including only those clusters including more pixels than a threshold value;

mean cluster area, i.e. total area divided by number of clusters;

mean cluster area for clusters larger than a predetermined threshold;

mean distance between centres of clusters, using the smallest of the distances from a first cluster to each of the other clusters as the distance for that cluster;

mean distance between clusters exceeding a predetermined size;

number of clusters; and

number of clusters exceeding a predetermined size;

or any combination of the above.

The above processing scheme can be applied to assay results generating more than one agglutinate type with each agglutinate type being of a different colour. In this case, a plurality of foreground colours, one corresponding to each agglutinate type are used and pixels are grouped as background or one of the foreground colours using a corresponding method to the above.

Other characteristics of the digital image, for example descriptive of the texture of the image, may be used to derive the quantified result, either with or without classifying the image into clusters. For example, these characteristics may include:

1: Standard deviation

2: Mean

3: Higher order statistical moments

4: Autocorrelation as described in Milan Sonka et al., Imaging Processing, Analysis and Machine Vision Chapman & Hall, 1993

5: Fourier spectrum as described in Milan Sonka et al., Imaging Processing, Analysis and Machine Vision Chapman & Hall, 1993

4: Fractal signature as described in F. Albregsten, Fractal Texture Signature Estimated by Multiscale LIT-SNN and MAX-MIN operators on LANDSAT-5 MSS Images of the Antartic Proceedings, 6th SCIA, pp. 995-1002, Oulo Finland, Jun. 19-22, 1989

5: LIT (local information transform) as described in R. M. Haralick, Statistical Image Texture Analysis, In Handbook of Pattern Recognition and Image Processing, Academic Press, 1986

6: GLDM (gray level difference method) as described in R. M. Haralick, Statistical Image Texture Analysis, In Handbook of Pattern Recognition and Image Processing, Academic Press, 1986.

These properties may be calculated from the red, green or blue components of the pixels or from a combination of two or more of these.

The chemical properties indicated by the assay result can then be calculated either by comparison with empirically derived data and interpolation or by an algorithm.

The PC at this stage should prompt the operator to identify the patient from whom the samples derive if this information has not already been supplied. This could be input manually, but desirably the PC will be linked to a bar code reader, such as an Opticon ELT 1000 wedge reader, so that patient codes may be read in from sample container labels.

The data can at this stage be exported, e.g. to the patient's physician's database or a central hospital computer. A preferred export format is the American Society for Testing and Materials (ASTM) format.

### EXAMPLE 1

An Avitex-CRP test kit from Omega Diagnostics Ltd, of Alloa, Scotland was used. The test kit contains white latex particles coated with antibodies to CRP, a positive and a negative control. The test is normally performed by application of one drop of latex suspension on a black plastic test slide, followed by one drop of sample (either patient serum or control), stirring with a wooden stirrer for two minutes, and inspecting the plate for visible aggregates.

We performed the test in a microtitre plate by mixing 25 microliter latex suspension with 25 microliter sample, followed by gentle stirring for two minutes. The microtitre plate was covered by a black plastic sheet and scanned in a Hewlett Packard Scan Jet 6100 C scanner connected to a PC.

The samples tested were a dilution series of the positive control enclosed with the kit. The scanner automatically identified the wells in the microtitre-plate where the reactions had occurred, and calculated the average Standard Deviation (SD) of the colours red, green and blue in an area of 3×3 mm about the centre of each well.

The results obtained where as follows:

| Sample | SD | Visual appearance |
|---|---|---|
| Undiluted (100%) | 11.0 | Large aggregates |
| Diluted 4 + 1 (80%) | 9.1 | Clearly visible |
| | | aggregates |
| Diluted 3 + 2 (60%) | 6.5 | Visible aggregates |
| Diluted 2 + 3 (40%) | 3.0 | Faintly visible |
| | | aggregates |
| Diluted 1 + 4 (20%) | 3.0 | No aggregates |
| Negative control | 3.0 | No aggregates |

A value of CRP is not stated for the positive control. However, the detection limit for the kit is stated to be 6 mg/L which seems to be between 30 and 40% dilution of the control. Thus, the control appears to be about 20 mg/L.

### EXAMPLE 2

To coat particles with antibodies, a 1 ml suspension containing 5.7% particles of amino-substituted, white polystyrene particles of average diameter 0.23 μm, available from Bangs Laboratories Inc. of Indiana, USA, was subjected to buffer change in a hollow fibre unit resulting in a final composition of 5% particles in 0.1 mol/l sodium borate buffer (pH 8.0) containing 0.02% NaN_{3. To} 1 ml of the suspension was added 20 μl of a solution containing about 2 mg/ml of rabbit polyclonal antibodies to human transferrin and incubated at 20° C. under end-over-end mixing for about 18 hours. The suspension was thereafter subjected to centrifugation sufficient to collect the particles in a pellet in a test tube, and free binding sites in the particles were blocked by resuspension in 1 ml 0.1 mol/l sodium borate buffer (pH 8.0) containing 0.033% human serum albumin and 0.02% NaN₃ (blocking medium), and incubation for two hours at 20° C. Thereafter, the suspension was subjected to two cycles of centrifugation sufficient to collect the particles in a pellet, and resuspension in 1 ml of 0.1 mol/l Tris-HCl-buffer (pH 7.4) containing 0.33% human serum albumin and 0.02% NaN₃ (washing medium) and centrifugation. Finally, the particles were suspended in 1 ml of the washing medium.

The standard serum Seronorm available from Nycomed Pharma of Oslo, Norway, containing 2.7 g/l Transferrin was diluted with 0.154 mol/l NaCl to yield a series of solutions containing 10, 20 and 40 mg/l of Transferrin, respectively. In addition, a blank containing no Transferrin was included.

The agglutination reaction was carried out as follows. 25 μl of the latex suspension was mixed with 25 μl of one of the Transferrin solutions on a horizontally positioned transparent plexiglass plate visualised against a dark, underlying surface, and mixed by circular rotations with a wooden stick to smear out the mixture over a circular surface with a diameter of about 1.5 cm. After about five minutes, visible agglutination took place in the solutions, except for the blank. Visual inspection of the agglutinates gave the following results:

| Transferrin | |
|---|---|
| concentration | Visual appearance |
| 40 mg/l | Clearly visible, large agglutinates |
| 20 mg/l | Clearly visible, moderately sized |
| | agglutinates |
| 10 mg/l | Faintly visible agglutinates |
| 0 mg/l | No visible agglutination |

The plexiglass plate was transferred to a Hewlett Packard ScanJet 6100c scanner and scanned at a resolution of 150 dpi. The pictures obtained were then subjected to the following numerical analysis methods (described in detail below) within a defined area of each agglutination pattern obtained:

Trimmed mean method, with variations in the High and Low exclusion limits (results not shown);

Standard deviation method, with variations in the filter size and the High and Low exclusion limits (FIG. 5);

Fractal Signature method, with variations in the filter sizes and the High and Low exclusion limits (FIG. 6);

High pass method, with variations in the filter sizes and the High and Low exclusion limits (FIG. 7); and

Colour Level Difference Method (CLDM) method, with variations in the filter size and the High and Low exclusion limits, and taking the CLDM mean (FIG. 8), CLDM energy (FIG. 9), CLDM contrast (FIG. 10), and the CLDM homogeneity (FIG. 11).

The results obtained applying an optimal combination of the variable parameters are shown in FIGS. 5 to 11. The curves clearly demonstrate a dose-dependent relationship illustrating that the agglutination reactions can be read quantitatively by applying a scanner and a suitable set of algorithms, whereas such reactions can only be read as a simple, qualitative yes/no-reaction by the prior method of visual inspection.

When the data are analysed by the Standard Deviation method, fairly linear dose response relationships are achieved over a range of filter sizes and exclusion limits. Thus, this method appears to be well-suited for analysis of a test with the present chemistry profile.

Applying data analyses by the Fractal Signature method demonstrates that the exclusion limits are of minor importance, and that similar dose response curves may be achieved with various combinations of filter sizes. The curve profiles are almost linear in the lower concentration range, and then level out. Thus, data analysis by Fractal Signatures can be suitable where analysis should be weighted to the lower part of the curve, and the upper parts play a less significant role.

The opposite conclusion is reached when the High Pass analysis method is applied. The method gives less ability to discriminate in the lower range, and is fairly linear in the upper. Thus, this method may be useful if a certain cut-off concentration is envisaged. The results are improved when lower exclusion limits are chosen.

Applying CLDM Mean to the analysis of the data gives a sigmoid dose response relationship and is thus weighted towards the middle part of the curve. The method requires rather low filter values, and is then less dependent on the exclusion limits.

A similar conclusion may be drawn from application of CLDM Energy and CLDM Homogeneity. The curve is sigmoid, and is best achieved at lower filter values. The dose-response relationship is negative.

Application of CLDM Contrast to the data analysis gives results resembling the High Pass method: Less ability to discriminate in the lower range, and an increasing dose response in the upper part. Thus, this method may also be suited if a certain cut-off value is desired.

The overall data demonstrate that agglutination may be measured by a obtaining a digital image using a scanner, and application of the resulting images to various mathematical/statistical analysis to arrive at a method that quantifies the result. The method of mathematical/statistical analysis may be selected to suit the particular features of the agglutination assay in question.

### EXAMPLE 3

To coat the particles with antibodies, a 1 ml suspension containing 5.7% particles of amino-substituted polystyrene particles of average diameter 0.23 μm, available from Bangs Laboratories Inc. of Ind., USA, was subjected to buffer change in a hollow fibre unit, resulting in a final composition of 5% particles in 0.1 mol/l sodium borate buffer (pH 8.0) containing 0.02% NaN₃. To the suspension was added 70μg of each of two anti-C-reactive protein (CRP) monoclonal antibodies (6405 and 6404 available from Medix Biochemica, Helsinki, Finland) and the suspension was then incubated at 20° C. under end-over-end mixing for about 18 hours. The suspension was thereafter subjected to centrifugation sufficient to collect the particles in a pellet in a test tube, and free binding sites in the particles were blocked by resuspension in 1 ml 0.1 mol/l sodium borate buffer (pH 8.0) containing 0.033% human serum albumin and 0.02% NaN₃ (blocking medium), and incubation for two hours at 20° C. Thereafter, the suspension was subjected to two cycles of centrifugation sufficient to collect the particles in a pellet, and resuspension in 1 ml of 0.1 mol/l Tris-HCl-buffer (pH 7.4) containing 0.33% human serum albumin and 0.02% NaN₃ (washing medium) and centrifugation. Finally, the particles were suspended in 1 ml of the washing medium. 8 μl of a solution of 25 mg/ml of human C-reactive protein (CRP), available from ICN Pharmaceuticals Inc. of California, USA, was added to 250 μl washing buffer to form a solution of 100 mg/l CRP. The solution was diluted in a series forming concentrations of 75, 50, 25, and 12.5 mg/ml CRP, respectively.

25 μl of the latex suspension was mixed with 25 μl of one of the CRP solutions on a horizontally positioned transparent plexiglass plate visualised against a dark, underlying surface, and mixed by circular rotations with a wooden stick to smear out the mixture over a circular surface with a diameter of about 1.5 cm. After about five minutes, visible agglutination took place in the solutions containing the highest concentrations of CRP.

Visual inspection of the agglutinates gave the following results:

| CRP | |
|---|---|
| concentration | Visual appearance |
| 100 mg/L | Clearly visible, large agglutinates |
| 75 mg/L | Clearly visible, large agglutinates |
| 50 mg/L | Clearly visible agglutinates |
| 25 mg/L | No visible agglutination |
| 12.5 mg/L | No visible agglutination |

The plexiglass plate was transferred to a Hewlett Packard ScanJet 6100c scanner and scanned at a resolution of 300 dpi. The digital images obtained were then subjected to the following numerical analysis methods within a defined area of each agglutination pattern:

Standard Deviation method, with variations in the filter size and the High and Low exclusion limits (FIG. 12);

High Pass method, with variations in the filter sizes and the High and Low exclusion limits (FIG. 13);

Fractal Signature method, with variations in the filter sizes and the High and Low exclusion limits (FIG. 14); and

Colour Level Difference Method (CLDM) method, with variations in the filter size and the High and Low exclusion limits, and taking the CLDM mean (FIG. 15), CLDM energy (not shown), CLDM contrast (not shown), and the CLDM homogeneity (not shown).

The results obtained applying an optimal combination of the variable parameters are shown in FIGS. 12 to 15. The curves clearly demonstrate that a dose-dependent relationship may be found by analyses of the pictures with the standard deviation, fractal signatures, high pass, and colour level difference mean methods. Suitable dose-response curves where found for certain sets of parameters illustrating that the agglutination reactions can be read quantitatively using a scanner and a suitable set of algorithms. Such reactions can only be read as simple, qualitative yes/no-reactions by the known method of visual inspection.

The Standard Deviation method results in a slightly sigmoid curve, but is reasonably suited for application over the entire range measured. The Fractal Signature method weights precision in the lower part of the concentrations measured, whereas the High Pass method weights precision in the upper part of the concentrations. The CLDM Mean forms a sigmoid curve weighting the middle part of the curve. In this particular experiment, CLDM Energy, Contrast and Homogeneity (curves not shown) were less suited because they demonstrated small variation between the two lower, and the three upper CRP-values, respectively.

### STATISTICAL/MATHEMATICAL ANALYSIS METHODS

The following methods were used to analyse the digital image of the agglutination assay generated by the scanner. In the description of each method, the variable I(x,y) (=R(x,y), G(x,y) or B(x,y)) represents the image array of red, green or blue pixel values (0-255 for 24-bit colour) corresponding to the pixels making up the image of a selected region of the result of the agglutination assay. Each method is therefore carried out three times: once on the image array (R(x,y), G(x,y) and B(x,y)) for each colour component of the image. In the final calculated value, the calculated values for each colour array are summed. If required, the contribution from any particular colour array may be reduced or omitted.

The variable size 1 represents the size (in units of length, such as millimeters) of one side of a square filter within which the pixel values are analysed. The variable size2 represents the size (in units of length) of one side of an additional square filter within which the pixel values may also be analysed. The variables a and b correspond to the lengths size1 and size2 converted to numbers of pixels in the image. Thus, the square region defined by setting the value of size1 (size2) is a square of a (b) pixels by a (b) pixels.

According to each analysis method, one or more mathematical/statistical operations are carried out on the image array I(x,y) in each of the three colours (R, G, B) to generate a series of processed values. A histogram (frequency against processed value) of the processed values is generated and a lower percentage (“Low” in the Figures) and a higher percentage (“High” in the Figures) is excluded from further calculation. Thus, for example if Low=25% and High=25%, data from the first and fourth quartiles of the histogram is excluded from further calculations, and only data from the second and third quartiles is used. This exclusion of data is intended to reduce the effect of noise on the results.

According to each method, the mean value of the processed values (with the lower and higher percentages of data excluded) is calculated for each set of processed values generated from the red, green and blue arrays of image data. The calculated property value for the particular method is generated by summing the red, green and blue mean values, although one or more of these values may be excluded from the calculated property value, if desired. Feasibly, a weighted sum of the property values from each of the red, green and blue image array could be used to generate the final property value.

Standard Deviation Method

According to the standard deviation method, the standard deviation of each colour component (red, green and blue) within the filter window of the image array is calculated. In the absence of agglutination, the picture is uniform with close to zero deviation. In the presence of agglutination, the variation within a given area increases.

According to this method, an area containing the agglutination pattern is selected and the pixels making up this region of the image are set as I(x,y) (in three colours). A filter window size, size1, is also selected and a corresponding pixel window size, a, is calculated. The colour components (R, G or B) which are to be used to calculate the property value are also selected, because depending on the colour of the agglutinates it may be more effective to use only some of the colour values.

The standard deviation of the pixel values within a filter window (axa) centred on each current pixel (x,y) is calculated and a standard deviation array Da(x,y) is thereby generated for each colour component of the image. For each colour component of the standard deviation array, a histogram of standard deviation values is generated and the Low percentage and the High percentage of data values are excluded from further calculation. The mean standard deviation value, mR,mG,mB, for each colour component is then calculated from the remaining data. The calculated standard deviation value, p, is given as the sum of the mean standard deviation values, mR,mG,mB, for those colour components which were initially selected, i.e. according to the following algorithm:

p=0

if R selected then p=p+mR

if G selected then p=p+mG

if B selected then p=p+mB

Fractal Signature Method

According to the Fractal Signature method, two operators, Maxa( ) and Mina( ), are used which respectively compute the maximum and minimum (R,G,B) pixel values (colour level values) inside a window of size a about the current pixel (x,y). A combination of these operators can be used to generate an array containing only pixels which are part of a cluster of dimensions less than a. The combination Maxa(Mina( )) removes all peaks, i.e. regions of high localised pixel values, in the image of size less than a, and the combination Mina(Maxa( )) removes all valleys, i.e. regions of low localised pixel values, in the image of size less than a. From an image array, I(x,y), a first structure array, Sa(x,y)=Mina(Maxa(I(x,y)))−Maxa(Mina(I(x,y))), representing clusters in the image that are less than a in size can be generated. A processed image array, Fa(x,y)=Mina(Maxa(Maxa(Mina(I(x,y))))), can also be generated to remove all the clusters of size less than a. Similarly, for a filter size b, which is larger than a, a second structure array, Sb(x,y)=Minb(Maxb(Fa(x,y)))−Maxb(Minb(Fa(x,y))), can be generated representing clusters in the remaining image that are less than b in size. The fractal signature, T(x,y) is given by T(x,y)=log(Sa(x,y)/Sb(x,y))/log(a/b).

Thus, a value for agglutination can be generated in a corresponding manner to the standard deviation method, but in this case the fractal signature array, T(x,y), is used to generate the histogram, rather than the standard deviation array, Da(x,y).

High Pass Method

According to the High Pass method, a mean operator, Meana( ), is used which computes the mean (R,G,B) pixel value inside a window of size a about the current pixel (x,y). The high pass array of pixel data is generated using two filter sizes, a and b, and is defined as Hab(x,y)=Abs(Meana(x,y)−Meanb(x,y)), where Abs represents the absolute value operator. The High Pass array therefore represents the degree of variation of the image array between the scale of the smaller filter, a, and the scale of the larger filter, b.

Thus, a value for agglutination can be generated in a corresponding manner to the standard deviation method, but in this case the high pass array, Hab(x,y), is used to generate the histogram, rather than the standard deviation array, Da(x,y).

Colour Level Difference Method (CLDM)

According to the Colour Level Difference Method of analysis, the (R, G or B) colour value of the current pixel is compared to the (R, G or B) colour value of each pixel which is a distance a from the current pixel.

Thus, the CLDM value is equal to Abs(I(x,y)−I(x′,y′)) for all pixels (x′,y′) which are at a distance a from the current pixel (x,y). Clearly, there are multiple CLDM values for each pixel as there are multiple neighbouring pixels and thus according to this method, a histogram of CLDM value (0 to 255, for 24 bit colour) is generated directly, without generating a processed value array. It will be seen therefore that the CLDM values provide an indication of the degree of colour variation in the image on the scale of the current filter size, a.

The histogram is normalised (each frequency value is divided by the total number of data items) and the Low and High percentages of data are discarded as with the preceding methods. Thus, for each colour component (R, G and B), a respective normalised histogram, h(i), is generated with the variable i representing the possible values of the colour level difference (0 to 255, for 24 bit colour). For each colour component any of the following parameters can be calculated by summing over all values of i:

(a) CLDM-Mean: v=Σi×h(i)

(b) CLDM-Energy: v=Σh(i)×h(i)

(c) CLDM-Contrast: v=Σi²×h(i)

(d) CLDM-Homogeneity: v=Σh(i)/(i+1)

The calculated CLDM value, p, is given as the sum of the CLDM parameter, vR,vG,vB, for those colour components which were initially selected for inclusion in the calculated value, i.e. according to the following algorithm:

p=0

if R selected then p=p+vR

if G selected then p=p+vG

if B selected then p=p+vB

Trimmed Mean Method

According to the trimmed mean method, for each colour component (R, G, B) of the image array, a histogram of colour level value, i.e. pixel value, is generated and the Low percentage and the High percentage of data values are excluded from further calculation. The mean colour level value, mR,mG,mB, for each colour component is then calculated from the remaining data. The calculated trimmed mean value, p, is given as the sum of the mean values, mR,mG,mB, for those colour components which are selected for inclusion in the result.

Thus, in this case no mathematical/statistical operation is carried out on the image arrays before the histogram is generated.

Although the present invention has been described in terms of a diagnostic system and method of applicability to the field of medical testing, it will be appreciated that the invention is of applicability in any field where a quantified result is required by analysis of an agglutination assay.

Furthermore, the invention has been described with particular reference to a personal computer. As will be understood from the foregoing, any general-purpose computer may be employed for the purposes of the invention and this is intended to be encompassed within the scope of the appended claims.

1. Apparatus for the analysis of an agglutination assay comprising:
an imaging device arranged to generate a digital image of an assay result comprising a mixture of a sample and at least one agglutination reagent; and
data processing means arranged to process said digital image to generate a quantitative result representative of the degree of agglutination of the sample and reagent.

2. Apparatus as claimed in claim 1, wherein the imaging device is a desk top, flat bed computer scanner.

3. Apparatus as claimed in claim 1 or 2, wherein the data processing means comprises a personal computer.

4. Apparatus as claimed in any preceding claim, wherein the digital image is a digital colour image.

5. Apparatus as claimed in any preceding claim, wherein the data processing means is arranged to locate automatically digital image data corresponding to said assay result within the digital image.

6. Apparatus as claimed in any preceding claim, wherein the data processing means is arranged to determine at least one statistical characteristic of the distribution of pixels within the digital image.

7. Apparatus as claimed in any preceding claim, wherein the data processing means is arranged to determine the proportion of an area of the digital image representative of agglutination products.

8. Apparatus as claimed in any preceding claim, wherein the data processing means is arranged to locate within the digital image clusters of contiguous pixels which are representative of agglutination products.

9. Apparatus as claimed in claim 8, wherein the data processing means is arranged to generate the quantitative result by reference to the area of the clusters.

10. Apparatus as claimed in claim 8 or 9, wherein the data processing means is arranged to generate the quantitative result by reference to the distribution of the clusters in the digital image.

11. Apparatus as claimed in any of claims 8 to 10, wherein the data processing means is arranged to generate the quantitative result by reference to the number of the clusters in the digital image.

12. A method for the analysis of an agglutination assay comprising the steps of:
generating a digital image of an assay result comprising a mixture of a sample and at least one agglutination reagent; and
processing said digital image to generate a quantitative result representative of the degree of agglutination of the sample and reagent.

13. A method for performing an agglutination assay comprising the steps of:
providing a sample;
providing at least two agglutination reagents, each having different optical properties;
mixing the sample and the reagents to form an assay result;
generating a digital image of the assay result; and
processing said digital image by reference to the optical properties of each reagent to generate a quantitative result representative of the degree of agglutination of the sample and each reagent.

14. A method as claimed in claim 13, wherein the optical properties are the colours of the reagents.

15. A method as claimed in any of claims 12 to 14, wherein the digital image is a digital colour image.

16. A method as claimed in any of claims 12 to 15, wherein the processing step comprises automatically locating digital image data corresponding to the assay result within the digital image.

17. A method as claimed in any of claims 12 to 16, wherein the processing step comprises determining at least one statistical characteristic of the distribution of pixels within the digital image.

18. A method as claimed in any of claims 12 to 17, wherein the processing step comprises determining the proportion of an area of the digital image representative of agglutination products.

19. A method as claimed in any of claims 12 to 18, wherein the processing step comprises locating within the digital image clusters of contiguous pixels which are representative of agglutination products.

20. A method as claimed in claim 19, wherein the processing step comprises generating the quantitative result by reference to the area of the clusters.

21. A method as claimed in claim 19 or 20, wherein the processing step comprises generating the quantitative result by reference to the distribution of the clusters in the digital image.

22. A method as claimed in any of claims 19 to 21, wherein the processing step comprises generating the quantitative result by reference to the number of the clusters in the digital image.

23. Computer software which when run on data processing means processes a digital image of an assay result comprising a mixture of a sample and at least one agglutination reagent, and generates a quantitative result representative of the degree of agglutination of the sample and the reagent in accordance with the method of any of claims 12 to 22.
